(19) United States
(12) Patent Application Publication
(10) Pub. No.: US 2024/0358942 A1
(43) Pub. Date: Oct. 31, 2024

(54) BREATHING ASSISTANCE DEVICE WITH CONTINUOUS POSITIVE AIRWAY PRESSURE

(71) Applicant: SEFAM, Neuilly-Sur-Seine (FR)

(72) Inventors: Bertrand POIROT, Varangeville (FR); Nicolas CLERC, Nancy (FR); Adrien SERIN, Saint-Max (FR); Jean-Sebastien LAMY, Ludres (FR)

(21) Appl. No.: 18/637,756

(22) Filed: Apr. 17, 2024

(30) Foreign Application Priority Data
Apr. 27, 2023 (FR) 2304287

(51) Int. Cl. A61M 16/00; A61M 16/06; A61M 16/08

(52) U.S. Cl. CPC A61M 16/0063; A61M 16/0875; A61M 2016/003; A61M 2205/42; A61M 2205/75

(57) ABSTRACT

A breathing assistance device with continuous positive airway pressure for the treatment of sleep-related breathing disorders includes an air inlet duct conveying an incoming air flow to a compressor. There is a compressed air flow at the outlet of the compressor connected to a pressurized air outlet duct to a mask applied to the respiratory tract of a patient. The air inlet duct opens into a compartment that is upstream-looking in the direction in which the air flows—from the inlet of the compressor, and has, at that location, a guiding device for the flow of air first toward a bottom wall of the compartment oriented substantially perpendicular to the direction of suction of the air in the compressor. The bottom wall is at least partially covered with a structure with rigid and non-deformable honeycomb patterns.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to the field of breathing assistance devices capable of exerting ventilation with continuous positive airway pressure (CPAP) to treat respiratory disorders that manifest in the form of obstructive apnea and/or hypopnea.

### 2. Description of Related Art Including Information Disclosed Under 37 CFR 1.97 and 37 CFR 1.98

The invention is intended to treat the effects of what it is commonly called OSAHS, for Obstructive Sleep Apnea/Hypopnea Syndrome, specifically in the light and deep sleep phases of people who are afflicted with it. It comes in the form of involuntary pauses in breathing resulting from a temporary and whole or partial closure of the respiratory tract, for example caused by a release of the tongue and throat muscles. It is now known that these disorders have a narrow link to the prevalence of other pathologies for which they are a risk factor, such as hypertension and cardiovascular diseases.

To overcome the disadvantages and risks of these temporary stoppages of the respiratory system, ventilation devices such as the one of the invention are used, to prevent or to remedy the blockage of said airways by creating a positive pressure therein, which holds them artificially open as soon as a deficit of pressure and/or flow rate is detected. These devices, referred to as continuous positive airway pressure (CPAP) devices, are electrical devices based on an air compressor, a pressurized air at a predetermined level being necessary to unblock the respiratory tract as soon as a closure is measured.

However, the problem immediately posed when designing a CPAP device is the sound level produced during its operation. It will readily be understood that these devices are used at night, in a process to assist in sleeping, and as such operating noise is counterproductive. The existence of such a noise may also constitute a disturbance factor on several levels: besides the inconvenience induced during the treatment, when a person accepts and uses the device, the sound nuisance can be dissuasive before treatment begins, leading to its refusal.

In practice, to make the device—and therefore the treatment-more acceptable, it is desirable to reduce as much as possible the overall sound level of the device, so as not to disrupt the sleep-filled nights of the users, and optionally to convince those who are reluctant that the sound nuisance is kept at a level that will not bother them.

One of the well-known noise attenuation means consists of the use of absorbent foams, positioned at appropriate locations of the machine. This may be away from the air flow generated in the device, for example in the pads placed under the device, to attenuate vibrations. The foam can also be arranged between the internal noisy members and external covers, etc. The solutions used may also be provided in the air flow, by producing, for example, all or part of the walls guiding said flow with foam. However, this arrangement proves problematic because the foams have airflow resistivity. Part of the kinetic energy of the air flow is dispersed in the foam and results, with equivalent driving geometry, in a loss of airflow performance. A second problem relates to patient safety. The absorbent foams may in fact, depending on their material and the conditions of use, fall apart after a certain time and/or prolonged exposure to moisture. In such a case, there is a real risk of detachment, and then movement of the elements detached with the air flow of the machine, and then ingestion of these particles by the patient. There is also a risk of chemical alteration of the material (by particular hydrolysis).

However, medical devices for the treatment of sleep apnea are subjected to strict regulatory requirements, with normative standards that change regularly. A manufacturer was once forced into a product recall due to complaints: the foams degraded and particles were ingested by the patients, several of whom suffered pain and headaches possibly related to this ingestion. Indeed, it is now known that certain materials of the foams degrade by hydrolysis and generate volatile organic substances (VOCs) incompatible with safety standards, as the possible chemical alteration of these particles after hydrolysis potentially makes them toxic. This actually depends on the material of the foam chosen to equip the device. It should be noted that the foams are more generally difficult to clean or disinfect.

Another technical problem encountered, although less so, falls under scaling production and relates to the method for assembling the finished product. The absorbent foams are generally soft and deformable. During the assembly of the device by the production operator, it is possible for a foam to be, depending on the complexity of the assembly, incorrectly positioned or deformed. This may have the consequence of degrading the sound and/or airflow efficiency of the duct.

The most effective means of reducing the final sound level of a product is to reduce the sound emission of the source nuisance elements. The problems of sound level in electromechanical devices arranging gas compression are of various origins, and the solutions to respond to them each have their limits. In CPAP devices, the main source of noise is the compressor, and the first possible action is to limit the transmissions of vibrations of the compressor to the rest of the device, but this then concerns only the “vibratory” part of the sound level, which may be damped by the chosen means. The other noise causes have their source in general in the airflow: this is all the friction and percussion of the gas flows which travel through the machine, and which are not reduced (or barely so) by the conventional means mentioned.

In order to reduce the sound emissions that originate from airflow, it is therefore logical to seek to reduce friction, recirculation, turbulence and changes in direction of the air flows in the tubes, ducts, and conduits. For example, it is sought to soften the angles and the curvatures on the path of the air in the device. However, these technical solutions are also only partially effective, and it is not possible to avoid considering the “vibratory” part.

### BRIEF SUMMARY OF THE INVENTION

The present invention proposes a solution that dispenses with traditional designs based on the use of absorbent foams acting as an acoustic damper, and on the contrary, based on the use of rigid element judiciously positioned and sized to effectively replace said foams. The design of these rigid elements and the choice of where to locate them has a double objective. They first produce sound attenuation, by their direct effect on the propagation of the sound wave in the air. They are then aimed at mechanical attenuation, since they are provided to reinforce the rigidity of the mechanical parts wherein they are implemented, which consequently become less likely to vibrate or to deform, and therefore to generate noise.

For these reasons, and for other results which will be described in more detail below, the breathing assistance device of the continuous positive airway pressure type for the treatment of sleep-related breathing disorders of the invention conventionally comprises an air inlet duct conveying an incoming air flow to a compressor, the flow of compressed air at the outlet of the compressor being connected to a pressurized air outlet duct intended for a mask intended to be applied to the respiratory tract of a patient. From this traditional basic diagram, the design of the invention proposes numerous arrangements locally providing solutions to the overall problem of sound nuisance.

As the main solution, according to the invention, the CPAP device is first such that the air inlet duct opens into a compartment that is upstream-looking in the direction in which the air flows—from the inlet of the compressor, and has, at that location, means for guiding the flow of air first toward a bottom wall of said compartment oriented substantially perpendicular to the direction of suction of the air in the compressor, at least said bottom wall being at least partially covered with a structure with rigid and non-deformable honeycomb patterns.

In brief, particular honeycomb patterns have been placed in this compartment upstream of the compressor, having a predetermined geometry, and lining a wall of the compartment. They are positioned in the device so that they are not removable therefrom, the solid material used making them non-degradable over time. The structure with honeycomb patterns is in this case oriented substantially parallel to the flow of air such that it is guided at the outlet of this portion of the inlet duct opening into the compartment, and the gas flow therefore passes frontally through these patterns by dispersing on the surface of the bottom wall of the compartment. From an airflow point of view, the honeycomb patterns make it possible to linearize the flow of air, and therefore make it less turbulent. However, it is the turbulences in an air flow that generate noise.

More specifically, according to a configuration specific to the device of the invention, the air inlet duct comprises, at the inlet of said compartment, a ramp followed by a spiral portion developing in the honeycomb patterns of the bottom wall, said spiral portion having a rounded hollow terminal shoe, centered in said bottom wall and located in line with the axis of the compressor. The incoming air flow is therefore directed toward the bottom of the compartment, along an arrival direction, which has been seen to be generally parallel to it, then returned in a direction perpendicular to the compressor inlet. This portion of the inlet duct makes it possible to channel the upstream flow under the compressor so that there is less head loss and the least possible turbulence. The orientation (here centripetal in the clockwise direction) of the spiral makes it possible to start the flow spinning before it reaches the compressor, and that it then undergoes an even greater spin in the same direction. This also contributes to a better channeling of the flow.

The honeycomb structure of the bottom is in fact positioned in the direct vicinity of the suction zone of the compressor, where the incoming air flow is the most turbulent and where certain frequencies of the sound emitted by the compressor are generated. In order to further improve the control of the linearity of the flow entering the compartment, the spiral portion is provided, upstream of the rounded shoe, with a separating rib which has the function of better channeling the flow of air and then decreasing the turbulence. The recess of the hollow terminal shoe is further closed by a curved peripheral guide that helps redirect at least part of the flow to the turbine.

Furthermore, preferably, the ramp and the spiral portion constitute the upper face of a block, the base of which comprises a structure with honeycomb patterns, which makes it possible, in addition to reinforcing the rigidity of said block, also to reduce the sound level, the mechanical stiffening helping to remove or reduce certain vibrations. This block is also preferentially manufactured separate from the compartment and secured in particular to its honeycomb bottom. The rest of the honeycomb bottom of the compartment may either be a separate part resting on the bottom of the compartment, or be made during the injection of the rest of the half-shell of the frame, and therefore be integral with said bottom.

According to a possible configuration of the invention, the CPAP device comprises a buffer space provided upstream of the compressor, between on the one hand a partition wall separating the compartment from the compressor, said wall being provided with an opening placed in line with the terminal shoe of the spiral portion, and on the other hand said compressor. This is in fact a “buffer” volume that is left to the air flow, between the moment when it exits the compartment and when it enters the compressor. This space helps reduce the sound level.

Furthermore, upstream of the ramp, the air inlet duct of the device may comprise two superimposed parallel sections, a first section connected to the air inlet of the device and a second section opening into the ramp, said sections being connected by a rounded portion in a U. The two sections are for example such that the second is placed above the first and comprises access to the ramp which descends toward the compartment, the latter then being situated at the same “lower” level as the first section.

The first section may comprise, over a sufficient length, a linearizer profile, consisting of a honeycomb pattern oriented in the direction of the flow. This structure is positioned downstream of the air inlet of the device, whereby the flow can potentially exit in a turbulent mode. Indeed, variations in cross-sections, orientation changes, and any singular elements (grilles, filters, shapes surrounding the duct) are just some of the obstacles to the laminarity of the flow.

Preferably, the second section may comprise a Pitot probe of which a base plate resting on a partition wall of the superimposed sections has a structure with honeycomb patterns which stiffens this part of the device and prevents it from vibrating. This structure with honeycomb patterns also makes it possible to limit the transmission of sound from the first section toward the second, and vice versa.

One of the two ends of the airflow circuit of the CPAP device, the outlet duct downstream of the compressor may be equipped with an end piece for attenuating vibrations and noise comprising an air circulation baffle in the form of a tube having at least one turn and developing in a front plane perpendicular to the direction of the air flow in the final portion of the outlet duct, the inlet and outlet mouths of said tube being substantially parallel to said direction.

The vibrations of the device of the invention as well as the noise of the compressor may actually be perceptible at the mask worn by the patient, hence the importance of eliminating them or at least significantly reducing them, by the means and features already described and those to come.

More specifically, the tube of this end piece for attenuating vibrations and noise is made from two parts of said piece attached to each other, a base and a facade oriented along said frontal plane, and in that at least the face of the base on which the facade is attached is covered with a structure with honeycomb patterns. It is the face oriented toward the outside of the base that is covered with these patterns, which make it possible to dampen the sound level slightly more.

At the other end of the airflow circuit, the air inlet duct is closed, at its inlet, by a filter-holder cover provided with a grille offset relative to the axis of the inlet duct, said cover being secured to a roof flaring from the inlet duct and conveying the air from the grille to said duct. The air flow therefore enters the device by passing through the grille-behind which one or more filters-preferably oriented perpendicularly to the axis of the duct are placed so that the incoming flow is parallel to said axis, then is driven in the direction of the inlet duct, which requires it to change direction and to take an orientation normal to the first orientation during the crossing of the grille. The air flow then performs a second, opposite turn, so as to return to the inlet duct direction, which is in this case and preferably the first section of the inlet duct. The height position of the air inlet by the grille of the device has undergone iterations. Indeed, if it is located too low relative to the inlet duct, the sound waves emitted from this zone can be reverberated on the support surface of the device (for example the nightstand or floor on which the machine is sitting while in operation), which constitutes an undesirable phenomenon.

According to one possibility, lateral walls perpendicular to the surface of the grille at the cover or the roof comprise inclined oblique blades, protruding from said lateral walls towards the center of the grille, forming a device called a “fish trap” arranged to prevent sound waves from going back to the inlet grille of the compressor frame. Moreover, the cover provided with the grille also has, on its internal face opposite the inlet duct, a structure with honeycomb patterns. It is therefore not a solid and flat wall that is positioned facing the departure of the duct, which would not have any effect in terms of attenuating the turbulence present in the incoming air flow.

In practice, each honeycomb-patterned structure comprises hexagonal cells, which in fact is a honeycomb structure. The sizing of said honeycomb patterns is subordinate to the fact that the thicknesses of the walls of the patterns are of the order of 1.5 mm maximum. The empty spaces may have dimensions of 3 to 4 mm between walls. In practice, the walls of the patterns must be sufficiently thick and dense to stiffen the structure on which they are positioned, without however generating plastic production defects (in particular breaks).

According to a more comprehensive view of the composition of the invention, the compressor is fixed in a frame comprising two superimposed half-shells separated by a flexible middle seal, the inlet and outlet ducts being at least partially shaped in said frame. More specifically, in connection with the configuration explained previously, a Pitot probe and a ramp block and a spiral portion of the inlet duct are in particular immobilized in and by said frame, between the flexible middle seal and the half-shells, the flexible middle seal forming a partition wall on the one hand between two superimposed sections of the inlet duct and on the other hand between the compressor and a compartment at the outlet of the ramp, said seal comprising an opening placed above the end of said spiral portion and under the inlet of the compressor.

In other words, when the device of the invention is placed on a horizontal plane, this configuration comprises in particular:
means for holding one or more filters forming a baffle at the inlet followed by two sections of horizontal ducts placed one above the other
then a curved descending track towards the bottom lined with honeycomb patterns of a compartment
surmounted by a compressor, the outlet duct of which is slightly curved,
and which opens into a noise-reducing end piece also having a baffle.

This is a configuration having an architecture strictly specific to the invention. Most of the technical features mentioned above, including the respective configurations of the different parts, and the existence of different areas with honeycomb patterns, implement both of the functions already mentioned:
Sound attenuation, by their effect on the propagation of the sound wave in the air; the wave is for example reflected in the walls with honeycomb patterns, which may lead to a cancellation (by phase opposition) or a local attenuation of the propagation of sound, and
Mechanical attenuation, since the hexagonal structures of the honeycomb patterns strengthen the rigidity of the mechanical parts which are housed, which stop vibrating or deforming, or reduce these phenomena that generate noise.

### BRIEF DESCRIPTION OF THE SEVERAL VIEWS OF THE DRAWINGS

Other aims and advantages of the present invention will become apparent throughout the following description relating to a configuration which is given only by way of an indicative example. The understanding of this description will be facilitated in particular with reference to the attached drawings.

FIG. 1 shows a perspective view of a continuous positive airway pressure device frame according to the present invention.

FIG. 2 shows an elevation view of the air inlet side.

FIG. 3 is a sectional view of the inlet part of the airflow circuit.

FIG. 4 shows, in perspective view, the compressor frame, without the upper half-shell of the frame and without the compressor.

FIG. 5a and FIG. 5b show a Pitot probe modified according to the invention, respectively in perspective and in a cross-section.

FIG. 6 shows in perspective view the configuration of the median flexible seal.

FIG. 7 shows a sectional view along a plane perpendicular to the sectional plane of FIG. 3, in the part of the inlet duct comprising a descending ramp.

FIG. 8 is a perspective view of the lower half-shell of the frame with the various elements resting therein.

FIG. 9 shows a sectional view along a plane perpendicular to the sectional plane of FIG. 7 in the compartment wherein the descending ramp emerges, in the absence of the upper half-shell.

FIG. 10a and FIG. 10b show, in perspective view, the end piece for attenuating vibrations and noise, positioned at the end of the outlet duct of the pressurized air.

### DETAILED DESCRIPTION OF THE INVENTION

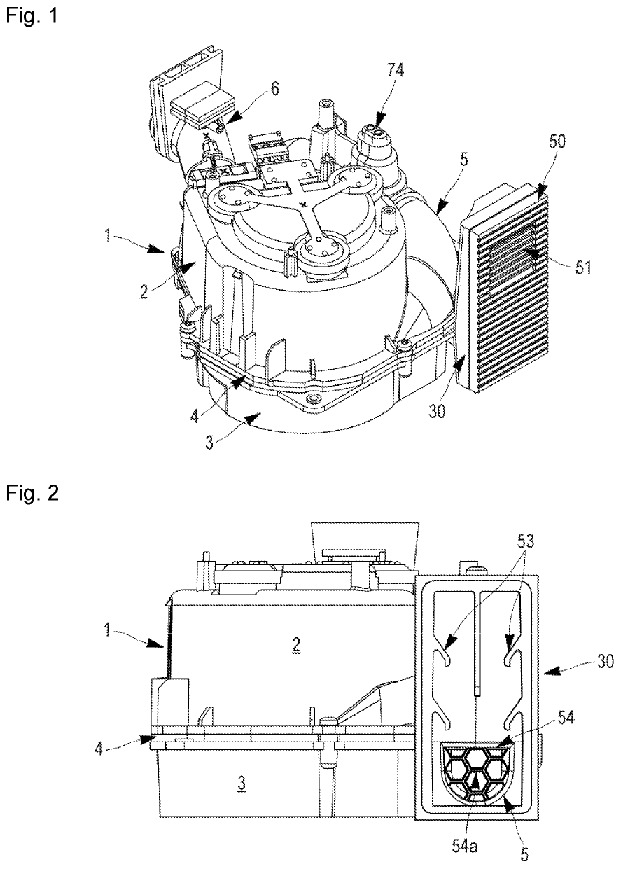

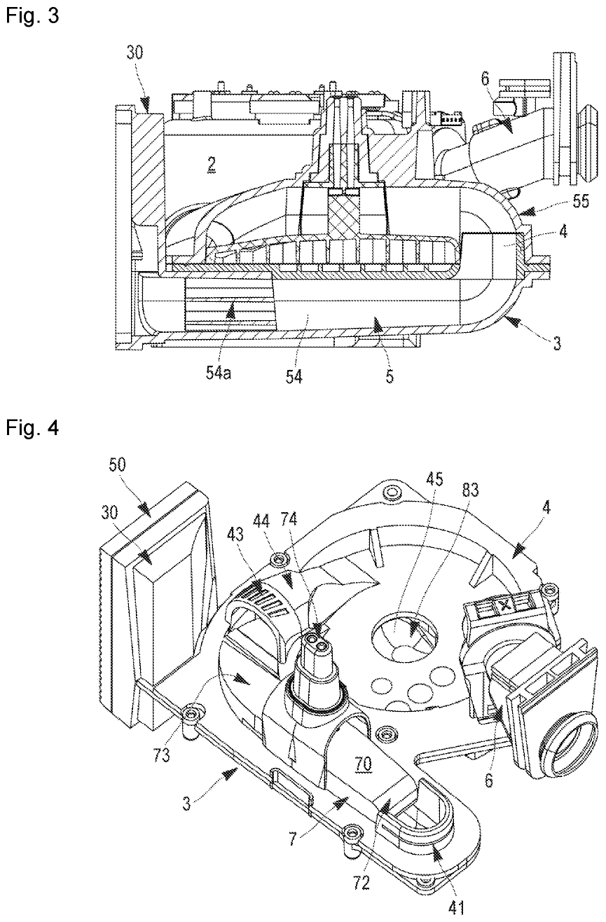

Referring to FIG. 1, the continuous positive airway pressure device of the invention comprises a frame 1 composed of two half-shells, respectively upper 2 and lower 3, separated by a flexible seal 4. An inlet filter-holder cover 50 comprising a wall provided with a grille 51 is arranged at the end of the inlet duct 5 described subsequently, attached to a mouth that widens into a roof 30 closed by said cover 50, said roof 30 being an integral part of the lower half-shell 3. At the other end of the internal airflow circuit to the device, an outlet duct 6 exits the frame 1 opposite the inlet filter part 50. The cover 50 provided with the grille 51 further comprises, on part of its interior face, a structure with honeycomb patterns, facing the end of the inlet duct 5. The cells form a stiffening relief located facing the start of a first section 54 of the inlet duct (see FIG. 3). The lateral walls of a shape perpendicular to the grille 51 of the filter-holder cover 50/roof 30 assembly comprise oblique blades 53 inclined downward. They are provided to “trap” the noise by preventing it from returning to the grille 51 and exiting it via this path. More precisely, these blades 53 project symmetrically from each lateral wall, and are oriented obliquely simultaneously towards the bottom of the cover 50 and towards the opposite lateral wall. One or more filters (not shown) are also positioned between these blades 53 and the grille 51, above the inlet of the duct 5.

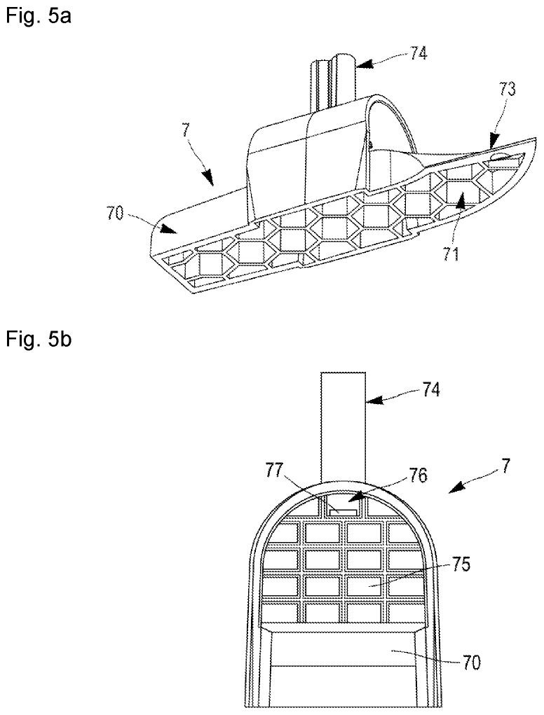

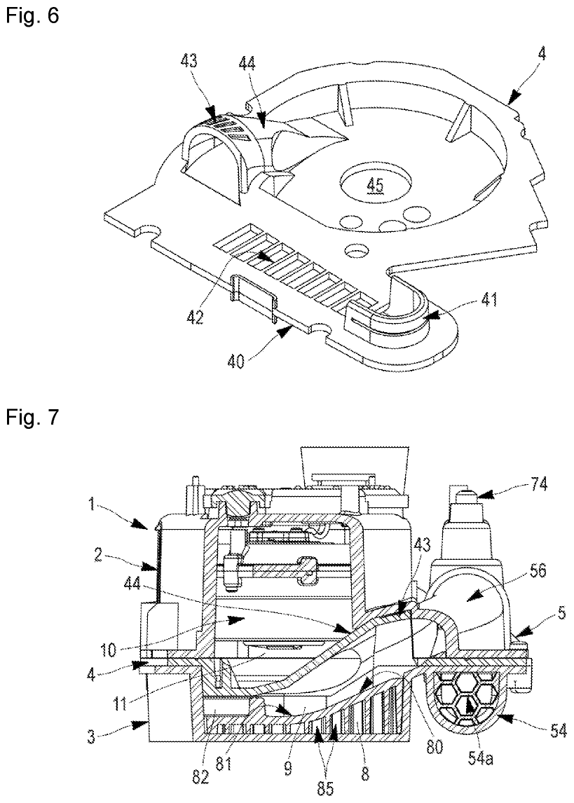

The flow of air passing through this inlet part of the airflow circuit changes direction after going through the grille 51, from horizontal to vertical, then changes again when moving towards the honeycomb structure placed at the bottom of the cover 50, which attenuates the effect of the flow turbulence, in order to engage horizontally in the first section 54 of the inlet duct 5 and pass through the linearizer profile 54a, the section of which has a honeycomb pattern (these mentions of horizontality and verticality refer to the figures, and should not be understood in absolute terms). A U-shaped portion 55 at the end of said first section 54 drives the flow in the opposite direction in a second section 56 located above the first. The two superimposed sections 54, 56 are in practice separated from each other by a part 40 of the flexible seal 4 (see FIG. 6), which comprises at this location a rounded wall 41 participating in the guiding of the half-turn of the flow in the U-shaped portion 55 arranged between the two sections 54, 56 and on the other hand by the base plate 70 of a Pitot probe 7 appearing in FIGS. 5a and 5b, provided with an inner grille 75 normal to the direction of the air flow (visible in FIG. 5b). The purpose of this part is to create a slight pressure difference between the upstream and downstream of the grille. This pressure difference is proportional to the flow rate through. A sensor (placed on top of the chimney 74 protruding from the upper half-shell 2 of the frame 1 upward) interprets this pressure delta into a proportional flow rate value.

The two sections 54 and 56 are thus formed by a subset of a small number of parts (here 4). The shape of the cross-section of these sections, as well as that of the portion 55, is U-shaped, called “semi-oblong”. It is particularly visible in FIG. 7, clearly showing the two superimposed sections 54, 56 arranged in an inverted U-shape since the U of the lower section 54 is arranged normally while the U of the upper section 56 is upside-down. This is the result of a compromise between, on the one hand, the number, arrangement, and mechanical functions of the parts forming this profile, and, on the other hand, the constraints specific to the plastic-forming technology of each of the parts (constant material thicknesses, draft angle, simplicity of design, etc.). To best linearize an air flow, the ideal passage section has a circular shape. The compromise presented here therefore consisted of approaching this shape as much as possible while keeping a floor (or a ceiling depending on which duct it is) flat to simplify the plastics processing and the assembly of the elements. The choice of this geometry is also based on digital simulations showing its relevance.

The Pitot probe 7 comprises a passage section for the air flow similar to the rest of the ducts upstream and downstream of said probe 7 (this section is therefore semi-oblong). It must ensure the primary flow function described above without generating an obstacle to the flow going through, to the point of creating pressure losses or undesirable turbulence. The passage section of the Pitot probe 7 comprises two main functional zones: the grille 75 with rectangular sections for most through which contribute to linearizing the flow and an obstructed section which forms an obstacle 76. The latter is more specifically constituted by an obstructive wall 76 and an opening 77 placed in the lower part of this obstructed wall 76. The opening 77 is particularly sized and particularly positioned on the air flow. It is upstream and downstream of the obstacle 76 that the measurements of pressures proportional to the flow through are carried out. Each of the two upper openings of the chimney 74 opens, for one, just a little upstream and for the other, just a little downstream of this obstructive wall 76. The sizing of these functional zones has been the subject of airflow and phonic performance and iterative digital simulations.

The base plate 70 of the Pitot probe 7 also comprises a structure with honeycomb patterns 71 which rests on a surface of the part 40 of the seal 4 provided with a particular design 42 with successive rectangular recesses (see in particular in FIG. 6). Above the honeycomb structure 71, the upper surface of the base plate 70 is configured to allow the smoothing of the guide of the air flow, provided in particular to prevent turbulence in the curves of the path taken by the air flow. Thus, the inlet end 72 of the base 70 is curved and forms an inner guiding surface of the U-shaped portion.

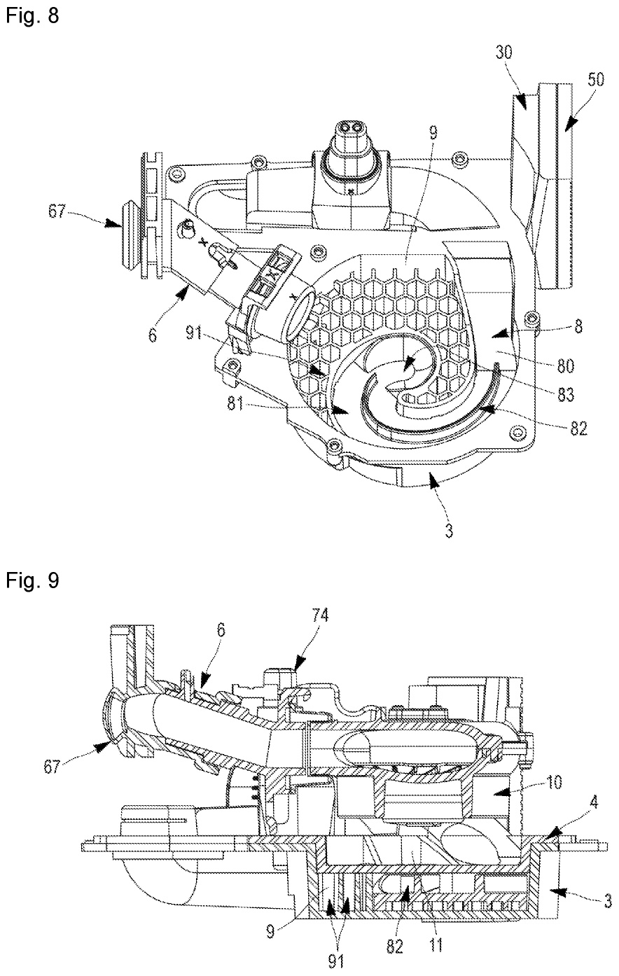

At the outlet of the Pitot probe 7, the outlet end 73 of the base plate 70 has a shape of starting a turn toward the next part of the inlet duct 5, comprising a ramp 80 descending toward a lower compartment 9. The latter is located, in the frame 1, at the same level as the first section 54 of the inlet duct. From said ramp 80, the wall which surrounds and delimits the inlet duct 5 no longer raises the half-shells 2, 3, as was at least partially the case until then. An arch 43 of the seal 4 followed by an inclined face 44 of this same seal 4 form the upper part of the duct, the lower track of which is raised by a block 8 (see in particular in FIG. 8) comprising the ramp 80 and a spiral portion 81 terminating in the bottom of the compartment 9. The base of this block 8 is also provided with a structure with honeycomb patterns 85. The spiral portion 81 is equipped with a separating rib 82 for fluidifying the air and attenuating the turbulence during the guiding of the air flow in the spiral.

The bottom of the compartment 9 also comprises a structure with honeycomb patterns 91. After the last turn of the spiral portion 81, the entire bottom is filled with honeycomb patterns 91, for which the dimension of the cells has undergone iterations until the satisfactory shape is found, in this case hexagonal.

This structure with honeycomb patterns 91 placed in the bottom of the compartment 9 is particularly important for the resolution of sound problems, due to its positioning in the immediate vicinity of the suction zone of the compressor 10, where the air flow is the most turbulent and where certain frequencies of the sound emitted by the compressor 10 are generated. It should be noted that this structure 91 can be manufactured independently, that is to say that it is a separate component placed in the bottom of the compartment 9 of the lower half-shell 3 of the frame 1 or, alternatively, injected with the rest of the half-shell 3 and therefore integrally attached to the frame 1.

The hollow and rounded terminal shoe 83 of the spiral portion 81 has a peripheral guide in the form of a spout which curves from the bottom of the central recess of the shoe 83 located in the extension of the spiral track 81 toward a rim located at the upper surface of the honeycomb patterns 91. This terminal shoe 83 is located under a circular opening 45 made in a central zone of the flexible seal 4 (see in particular in FIG. 6). When the air arrives at the end of the rib 82 of the track of the spiral portion 81, at the terminal shoe 83, it is just below the compressor 10, ready to be sucked.

As is particularly clearly apparent in FIG. 9, the compressor 10 is located above the compartment 9, from which it is separated by the flexible seal 4. It does not rest on said seal 4, a buffer space 11 having been provided for the incoming air flow, located between the location of its outlet from the lower compartment 9, and the location where it enters the turbine. The existence of this space 11 is very important for reducing the sound level of the device of the invention. The compressor 10 is substantially centered above the opening 45.

Once compressed in the compressor 10, the air flow is sent through an outlet duct 6 (see in particular in FIG. 9) before leaving the frame 1. Its dimensions and its curvatures have been optimized, based on numerical simulations, to attenuate the noise and vibrations as much as possible.

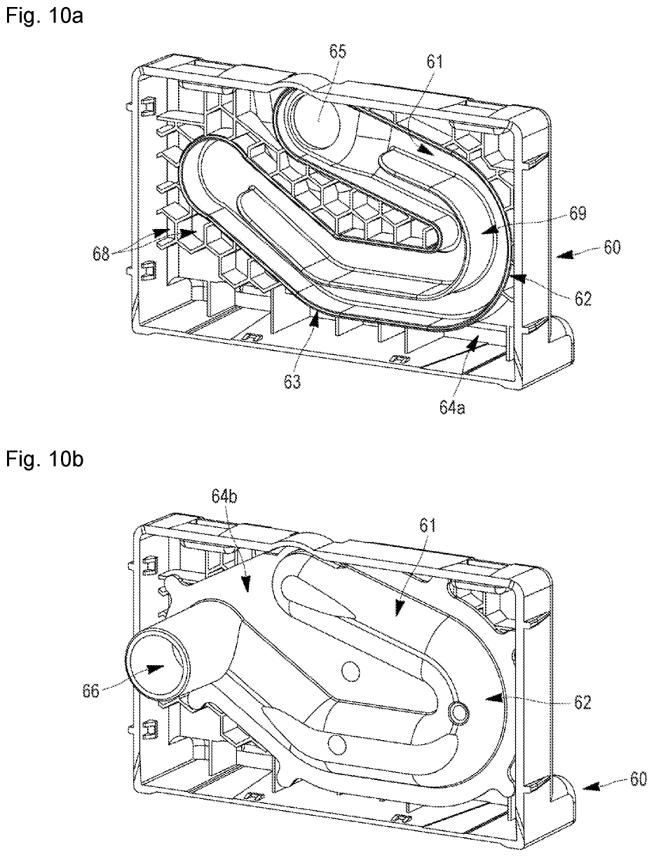

Finally, before leaving the device, the airflow passes through a final piece: this is an end piece 60 for attenuating vibrations and noise, shown in FIGS. 10a and 10b, in fact comprising a baffle placed in the circulation path of the air. The baffle takes the form of a tube 61 made from two parts of said piece 60 attached to each other, a base 64a and a facade 64b, the tube 61 created between them developing in a front plane perpendicular to the direction of the air flow at the outlet of the outlet duct 6. The length traveled by the flow in this tube 61, as well as the shape and dimensions of the cross-section through the passage of the flow, participate in the efficiency of the attenuation of vibrations and noise. A separating rib 69 protruding from the base 64a allows the fluidization of the air and the attenuation of the turbulences during the guiding of the air flow in the tubing 61, leading to a reduction of noise. More precisely, the baffle has a first turn 62 in a half-turn followed by a lighter turn 63 which reveals the direction of the tube 61 so that the inlet 65 and outlet 66 openings respectively of said tube 61 are almost at the same place. These two mouths 65 and 66 are parallel to one another and to the direction given to the flow at the end of the outlet duct 6. There, that duct has a fitting 67 for fastening to the inlet mouth 65 of the end piece 60.

The visible face of the base 64a is covered with a structure with honeycomb patterns 68. This end piece 60 adds a piece of additional duct to the airflow circuit of the device of the invention described until here, which serves to attenuate the sound level emitted downstream of the compressor 10, and particularly the noise specifically felt in the mask worn by the patient on their face.

The technical solutions to the problem initially posed, namely to reduce and/or eliminate the noise and vibration of the device when possible, are addressed without resorting to foam. Instead, all the features described above for acoustic attenuation are implemented by “rigid” pieces of the sort normally used to form the ducts of the air flow, these features being embedded in the shapes given to certain elements, in “anti-turbulence” ribs, in honeycomb structures located in certain volumes to be filled, etc., whose dimensions for attenuating the sound level have been the subject of iterative work to be correctly established.

The examples of the configuration of the CPAP device which appear in the figures should not be considered as limiting the invention, which on the contrary encompasses, for example, alternative forms in the ducts, honeycomb structures, inlet and outlet pieces, etc.

We claim:

1. A breathing assistance device with continuous positive airway pressure for the treatment of sleep-related breathing disorders, the device comprising:
a compressor;
an air inlet duct conveying an incoming air flow to the compressor;
a pressurized air outlet duct connected to the compressed air flow at the outlet of the compressor, being connected to a mask intended to be applied to the respiratory tract of a patient,
wherein the air inlet duct opens into an upstream compartment in the direction of circulation of the air flow from the inlet of the compressor, and
wherein the air inlet duct is comprised of, at that location, means for guiding the air flow first to a bottom wall of said compartment oriented perpendicular to the direction of suction of the air in the compressor, at least said bottom wall being at least partially covered with a rigid and non-deformable structure with honeycomb patterns.

2. The breathing assistance device with continuous positive airway pressure according to claim 1, wherein the air inlet duct comprises, at the inlet of said compartment, a ramp followed by a spiral portion developing in the honeycomb patterns of the bottom wall, said spiral portion having a rounded hollow terminal shoe, centered in said bottom wall and located in line with the axis of the compressor.

3. The breathing assistance device with continuous positive airway pressure according to claim 2, wherein the spiral portion is provided, upstream of the rounded shoe, with a separating rib.

4. The breathing assistance device with continuous positive airway pressure according to claim 2, wherein the ramp and the spiral portion constitute the upper face of a block, the base of which comprises a honeycomb structure.

5. The breathing assistance device with continuous positive airway pressure according to claim 2, further comprising: a buffer space provided upstream of the compressor, between a partition wall separating the compartment from the compressor, said wall being provided with an opening placed in line with the terminal shoe of the spiral portion, and said compressor.

6. The breathing assistance device with continuous positive airway pressure according to claim 2, wherein, upstream of the ramp, the air inlet duct comprises two superimposed parallel-shaped sections, a first section connected to the air inlet of the device and a second section opening into the ramp, said sections being connected by a rounded U-shaped portion.

7. The breathing assistance device with continuous positive airway pressure according to claim 6, wherein the first section comprises a linearizer profile being comprised of a honeycomb pattern oriented in the direction of the flow.

8. The breathing assistance device with continuous positive airway pressure according to claim 6, wherein the second section comprises a Pitot probe, a base plate of which resting on a partition wall of the superimposed sections has a honeycomb pattern structure.

9. The breathing assistance device with continuous positive airway pressure according to claim 1, wherein the outlet duct downstream of the compressor is equipped with an end piece for attenuating vibrations and noise comprising an air circulation baffle in the form of a tube having at least one turn and developing in a front plane perpendicular to the direction of the air flow in the final portion of the outlet duct, the inlet and outlet vents of said tube being substantially parallel to said direction.

10. The breathing assistance device with continuous positive airway pressure according to claim 9, wherein the tube of the vibration and noise attenuation end piece is comprised of two parts of said piece attached to each other, a base and a façade oriented along said front plane, and wherein at least the face of the base on which the façade is attached is covered with a structure with honeycomb patterns.

11. The breathing assistance device with continuous positive airway pressure according to claim 1, wherein the air inlet duct is closed, at its inlet, by a filter-holder cover provided with a grille offset relative to the axis of the air inlet duct, said cover being secured to a roof which flares from the inlet duct, conveying the air from the grille to said duct.

12. The breathing assistance device with continuous positive airway pressure according to claim 11, wherein lateral walls perpendicular to the surface of the grille at the cover or roof comprise inclined oblique blades, protruding from said lateral walls towards the center of the grille.

13. The breathing assistance device with continuous positive airway pressure according to claim 12, wherein the cover provided with the grille has, on its internal face opposite the inlet duct, a structure with honeycomb patterns.

14. The breathing assistance device with continuous positive airway pressure according to claim 1, wherein each structure with honeycomb patterns comprises honeycomb hexagonal cells.

15. The breathing assistance device with continuous positive airway pressure according to claim 1, wherein the compressor is attached in a frame comprising two superimposed half-shells separated by a flexible middle seal, the inlet and outlet ducts being at least partially shaped in said frame.

16. The breathing assistance device with continuous positive airway pressure according to claim 15, further comprising: a Pitot probe and a ramp block and a spiral portion of the inlet duct are immobilized in said frame, between the flexible middle seal and the half-shells, the flexible middle seal forming a partition wall between two superimposed sections of the inlet duct and between the compressor and a compartment at the outlet of the ramp, said joint comprising an opening placed above the end of said spiral portion and under the inlet of the compressor.
